(19) United States
(12) Patent Application Publication
(10) Pub. No.: US 2020/0345874 A1
(43) Pub. Date: Nov. 5, 2020

(54) FOOTWEAR SANITIZING DEVICE AND SYSTEM AND METHOD FOR USING SAME

(71) Applicant: James H. Barr, III, Jupiter, FL (US)

(72) Inventors: James H. Barr III, Jupiter, FL (US); Jason Franklin, Atlanta, GA (US)

(21) Appl. No.: 16/853,748

(22) Filed: Apr. 20, 2020

Related U.S. Application Data
(60) Provisional application No. 62/835,593, filed on Apr. 18, 2019.

(51) Int. Cl. A61L 2/07; A47L 23/02; A61L 2/24; A61L 2/20

(52) U.S. Cl. CPC A61L 2/07; A47L 23/02; A61L 2/24; A61L 2/20; A61L 2202/14; A61L 2202/11; A61L 2202/15; A61L 2202/17; A61L 2202/26

(57) ABSTRACT

Disclosed is a sanitizing device, system and method of sanitizing objects, such as, for example an article of footwear. In further aspects, the present disclosure relates to a sanitizing device, system and method of sanitizing objects configured such that it sanitizes the base or surface of an article of footwear while a user is standing on the device. In another exemplary aspect, the disclosure relates to a steaming apparatus, system and method of sanitizing objects configured such that it utilizes a steamed chemical to clean the underside of an article of footwear while a user is standing on the apparatus. In further aspects, the disclosure also relates to a steaming apparatus, system and method of sanitizing objects configured such that a chemical may be used in combination with steam to clean the underside of an object or article of footwear while a user stands on the apparatus. Disclosed is a method comprising: inserting a chemical disinfectant into a steaming apparatus; placing an object onto the steaming apparatus; activating the steaming apparatus; and sanitizing the object using a combination of the chemical disinfectant and steam.

### RELATED APPLICATIONS

This application claims the benefit of priority to U.S. Provisional Patent Application No. 62/835,593, filed on Apr. 18, 2019, the contents of which is incorporated herein by reference in its entirety.

### FIELD OF DISCLOSURE

The present disclosure relates to methods, systems, and devices for sterilizing various surfaces and articles, for example, footwear. Further, the present disclosure relates to methods and devices for disinfecting or sterilizing materials, surfaces or articles.

### BACKGROUND OF THE DISCLOSURE

In some situations, not properly cleaning your footwear can be hazardous to your health. For example, shoes and footwear track countless germs, mold, dirt, and bacteria into an individual's home, office, or dwelling. Thus, the conventional strategy is to have a mud room or door mat that allows for wiping off the bottom of an individual's shoes prior to entering a dwelling. This often causes problems because while the conventional strategy does prevent the tracking of visible traces of mud, dirt, water, or other liquids throughout the floors of the dwelling, it does not remove mold, viruses, bacteria, and other potential hazardous waste. For example, traces of mold, fecal matter, bacteria, and other matter or residues may remain even after vigorously wiping one's shoes and footwear on a placemat.

Accordingly, there remains a need for improved devices and methods for sanitizing surfaces, objects and footwear. This need and other needs are satisfied by the various aspects of the present disclosure.

### SUMMARY OF THE DISCLOSURE

In accordance with the purposes of the disclosure, as embodied and broadly described herein, the disclosure, in one aspect, relates to a sanitizing device, system and method of sanitizing objects, such as, for example an article of footwear. In further aspects, the present disclosure relates to a steaming apparatus, system and method of sanitizing objects configured such that it sanitizes the base of an article of footwear while a user is standing on the apparatus.

In another exemplary aspect, the disclosure relates to a steaming apparatus, system and method of sanitizing objects configured such that it utilizes a steamed sanitizing agent to clean and sanitize a surface, such as the underside of an article of footwear while a user is standing on the apparatus.

In further aspects, the disclosure also relates to a steaming apparatus, system and method of sanitizing objects configured such that a chemical may be used in combination with steam to clean the underside of an object or article of footwear while a user stands on the apparatus.

Additional aspects of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate several aspects of the disclosure and together with the description, serve to explain the principles of the disclosure.

FIGS. 1A-1D show depictions of views of a sanitizing device in accordance with an exemplary embodiment of the present disclosure.

FIG. 2 shows a depiction of a sanitizing device in accordance with an exemplary embodiment of the present disclosure.

FIGS. 3A-3B depict an upper housing frame of a sanitizing device in accordance with an exemplary embodiment of the present disclosure.

FIGS. 4A-4B depict an upper housing frame of a sanitizing device in accordance with an exemplary embodiment of the present disclosure.

FIGS. 5A-5B depict a sanitizing platform of a sanitizing device in accordance with an exemplary embodiment of the present disclosure.

FIG. 6 shows a flow diagram of a sanitizing device, system and method of sanitizing objects in accordance with an exemplary embodiment of the present disclosure.

### DETAILED DESCRIPTION OF THE DISCLOSURE

The present disclosure can be understood more readily by reference to the following detailed description of the disclosure and the Examples included therein.

Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

All publications mentioned herein are incorporated herein by reference to disclose and describe the methods and/or materials in connection with which the publications are cited.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this disclosure belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the disclosure. Thus, if there are a variety of additional steps that can be performed it is understood that each of these additional steps can be performed with any specific aspect or combination of aspects of the methods of the disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result.

### Sanitizing Devices and Systems

As briefly described above, the present disclosure relates, in various aspects, to a sanitizing device, system and method of sanitizing objects, such as, for example an article of footwear. In further aspects, the present disclosure relates to a steaming apparatus, system and method of sanitizing objects configured such that it sanitizes the base of an article of footwear while a user is standing on the apparatus.

In another exemplary aspect, the disclosure relates to a sanitizing device, system and method of sanitizing objects configured such that it utilizes a steamed chemical to clean the underside of an article of footwear while a user is standing on the apparatus.

In further aspects, the disclosure also relates to a steam-activated apparatus, system and method of sanitizing objects configured such that a chemical may be used in combination with steam to clean the underside of an object or article of footwear while a user stands on the apparatus.

In one aspect, the disclosure provides a sanitizing device for dispensing s sanitizing agent, such as steam and/or a chemical. In further aspects, the device may comprise a steam source; a sanitizing platform and switch in operable communication with the steam source, the switch configured to selectively dispense a predetermined volume of steam in a stream. In still further aspects, the switch may be a step-activated switch that is configured to activate the device when a user steps on the sanitizing platform. In yet further aspects, the device may comprise a container or compartment comprising sanitizing agent, the container being in operable communication with the steam source and/or stream. In some aspects, the steam stream may be effective to cause at least a portion of the sanitizing agent within the container to be drawn or pulled into the steam.

In further aspects, the switch may be configured to control a flow rate of steam flowing from the steam source (or a supply line in fluid communication with the steam source). In still further aspects, the switch may be configured to control flow by moving a valve position from a closed position to an open position. In yet further aspects, the switch may comprise an actuating mechanism. In some aspects, the switch comprises a step-triggered switch. In further aspects, the switch may be in operative communication with a valve for selectively moving the valve from at least one of: a closed position to an open position and an open position to a closed position to control flow from a steam source and/or sanitizing agent source.

In further aspects, the device may comprise a housing or casing for housing one or more device components. In still further aspects, the housing or casing may be configured for containing the steam source and/or sanitizing agent container. In yet further aspects, the housing can be any shape or sized, and preferably conforms to most feet. For example, various areas may be substantially flat or only slightly convex so to conform to footwear and feet to which the device may be in contact with. The housing can be any shape, and preferably is in the shape of a three-dimensional polygon and the housing walls define an interior space or interior sections for containing the operating elements of the invention. Any other shape (as used herein, the term shape is used in the broad sense of three-dimensional works) may be employed, so long as the shape is large enough and structured so as to be able to contain the various working components of the invention as more fully disclosed herein. The housing may be a generally hollow structure sized to contain a steam source and/or a sanitizing agent container. In yet further aspects, the device components can be all housed within the housing such that they are protected from, for example, for inadvertent shutting off or the environmental elements. In even further aspects, the housing can be comprised of plastic, plastic composite, reinforced plastic, metal, metal composite, or combinations thereof. In still further aspects, the plastic can comprise polypropylene, impact resistant plastic, or the like.

The switch can be secured to the housing at any convenient position where it may readily be actuated, for example, by stepping on the sanitizing platform. In further aspects, the switch may be mechanically connected in a known manner between the sanitizing platform and steam source to control the steam dispensed into a stream and through the platform. A switch can be a common on/off switch, such as a toggle, lever, push-button, or other switch. The disclosure herein contemplates any type of switch that would be practical with activating a steam source. As shown in illustrative embodiments, the switch may be a push button switch. In various aspects, the device comprises a switch in operative communication with a valve for selectively moving a valve from at least one of: a closed position to an open position and an open position to a closed position. In further aspects, the switch can be configured to operate a valve to allow a predetermined flow of steam through the valve. In yet further aspects, the switch can be in electrical and/or mechanical communication with the valve. In even further aspects, the switch can be configured to move the valve from the closed position to the open position when pressed. In other aspects, the valve can comprise any valve having an integral mechanical component capable of actuating the valve.

In further aspects, a housing may further comprise on opening through a wall of the housing for providing an amplifier on the outer surface of the housing that is connected to a control element or a sound element contained within the housing. A housing may further comprise on opening through the housing for providing a light, such as an LED light, on the outer surface of the housing that is connected to a control element, a fluid-level element, or a timing element contained within the housing. A light (and/or sound) may be turned on when sanitation or steaming is initiated and turned off when power to the steam source is turned off. Alternatively, powering on the steam source may also power on a timing element, and optionally a light (and/or sound), so that when a desired time period has occurred, the timing element may turn off the light (and/or sound), or may turn off a light (and/or sound) and the steam source, or the timing or control element may turn on sound or light after a period of dispensing steam. Alternatively, the timing element may be under a control that is separate from a control for the steam source. Various device components, such as switches, control elements, such as a polycarbonate circuit board and the programming to accomplish the disclosed activities and others, and elements such as timing elements, sound elements and lights, are known, and can be selected or commercially acquired by those of skill in the art. Wires for connecting the elements within the housing or on the surface are contemplated by the present invention.

In further aspects, the sanitizing platform of the device may further comprise a comprise a cleaning pad, such as a cleansing cloth or microfiber pad, that can be positioned on top of the sanitizing platform and can be held in place by a bracket, friction, adhesives, fasteners, fitting, or a closure. For example, the cleaning pad may be positioned directly over the apertures or openings of the sanitizing platform where the steam and/or sanitizing agent is dispensed. In further aspects, a sanitizing platform may be shaped to provide an application area that is in contact with a surface so that substantially all of the application area contacts the surface. For example, a sanitizing platform may be flat or convex in shape on at least its proximal side so that the application area, which may be a perforated surface, is shaped so that substantially all of the proximal side of the sanitizing platform contacts the surface, for example the surface of footwear. When an article of footwear is placed on the application area of the sanitizing platform, substantially all of the surface of the application area contacts the footwear receives steam and/or sanitizing agent therethrough (the application area).

In further aspects, the device may comprise a container, such as a liquid and/or sanitizing agent container, that can be positioned within a container pocket and can be held within pocket by a bracket, friction, adhesives, fasteners, fitting, or a closure on mouth or opening of container. For example, the container may be screwed in place. A steam source container and/or sanitizing agent container can be placed within housing during manufacture or at any time after manufacture. In further aspects, while the steam source container and/or sanitizing agent containers described herein can be permanently mounted in or on the device housing or device structure, this is not a requirement. For example, the steam source container and/or sanitizing agent container can be a cartridge or tank configured to be removably mounted in or on a portion of the device, e.g., to allow interchange and/or replacement of the steam source and/or sanitizing agent container. Such configurations allow users or others to select desired sanitizing agent compositions and sanitizing properties or levels in the device, e.g., for customization purposes, for personal preferences, to match desired use, environmental characteristics, or the like. To this end, the housing may use an ingress and egress element, as various embodiments of the steam source and/or sanitizing agent container may require exchanging or replacement on occasion.

In further aspects, the device may comprise at least one supply line configured to contain and/or carry the steam and/or sanitizing agent. In yet further aspects, the device may comprise a plurality of supply lines, for example, a steam supply line configured to contain and/or carry the steam and a sanitizing agent supply line to carry sanitizing agent. In some aspects, the device may comprise at least one sanitizing agent supply line configured to carry sanitizing agent from within the sanitizing agent container into a steam stream. In other aspects, the device may comprise at least one sanitizing agent supply line configured to carry sanitizing agent without steam from within the sanitizing agent container through the apertures of the platform. In further aspects, a steam supply line may be in fluid communication with the steam source. In still other aspects, the sanitizing agent supply line may be in fluid communication with the sanitizing agent container and/or steam stream. In still further aspects, the sanitizing agent supply line may extend into the sanitizing agent container interior space and which has at least one intake opening for intake of sanitizing agent stored in the sanitizing agent container to convey the sanitizing agent through one or more supply lines into the steam stream.

In further aspects, the cleaning pad may be detachably connected to the sanitizing platform. The cleaning pad can be any conventional cleaning material capable of cleaning or removing debris, dirt, soil, and contaminants from the surface of footwear. In further aspects, the cleaning pad may be made porous or otherwise made of a material that permits steam to effectively pass through and contact the bottom of a user's footwear. In some aspects, the cleaning pad may be removably mounted to allow interchange and/or replacement of the cleaning pad. Such configurations allow users or others to select desired cleaning pad materials compositions and sanitizing properties, washing the cleaning pad, and/or disposing the cleaning pad.

In various aspects, steam source can be any conventional steam source or system or apparatus for producing steam. It is only important that the steam be able to deliver a steam flow to the subject at a sufficient level to produce the effect intended in the disclosed methods, such as to sanitize and/or disinfect the article on the platform. In further aspects, the steam source may comprise at least one of a vessel, bottle, cylinder, tank, and cartridge, and the like, for containing the liquid, such as water or sanitizing agent, used to produce the steam. In yet further aspects, the steam source may be contained with the device housing. In still further aspects, the steam source may be detachably connected to the device, or otherwise replaceable. In some aspects, the steam source may an external steam source remotely located from the device. To this end, the device may comprise at least one container that may be detachably connected to the device. The container can be any conventional container, such a cartridge or tank, capable of storing and transferring liquid and/or sanitizing agents. In still further aspects, the container may be a replaceable cartridge or tank or the like. In further aspects, the sanitizing agent can comprise a liquid and/or solution. In even further aspects, the sanitizing agent can comprise one or more of: water, steam, a disinfecting composition, antimicrobial composition, antibacterial composition, bactericidal composition, bacteriostatic composition or the like. In some aspects, the sanitizing agent may be all natural, biodegradable, and/or no-toxic. In other aspects, the sanitizing agent may further comprise one or more fragrances. In further aspects, the sanitizing agent may be configured to kill at least 99% of microbes, mold, and bacteria, for example, at least 99.9 or 99.99%.

In various aspects, the device may comprise a flow control component for controlling steam and/or sanitizing agent flow. In further aspects, the flow control component can comprise a flow control orifice, or a pressure compensating or pressure regulating orifice. In yet further aspects, the flow control component is positioned after the valve opening in a flow passage. In still further aspects, the flow control component can be preconfigured at a fixed flow rate or configured to allow adjustment within the flow passage to control the flow rate. In yet further aspects, the device can further comprise a flow meter configured to measure a flow rate of the steam and/or sanitizing agent through a valve opening, flow control component, and/or outlet. In further aspects, the flow control component may be in fluid communication with the steam stream, and configured to control a flow rate of a mixed steam/sanitizing agent.

In still further aspect, the components can be integrally or mechanically attached to other components. In a yet further aspect, the disclosed components can be connected, attached, or mounted using a connecting means, the connecting means comprising a fitting, insert, adhesive, brazing, soldering, welding, spot weld, screw with nut, rivet, fitting, insert, threading, friction fit, or snap-fit or a combination thereof. In further aspects, the device components can be attached using a connecting means. In still further aspects, the connecting means can comprise a fitting, insert, adhesive, brazing, soldering, welding, spot weld, screw with nut, rivet, threading, friction fit, snap-fit, twist-lock, or interlocking mechanism or a combination thereof. In yet further aspects, the connection can be achieved using a snap, friction fitting, snap ring, O-ring, pressure fitting, clip, clasp, and the like. The snap ring or O-ring can be retained within a groove to accommodate the snap ring or O-ring. In a further aspect, the device can comprise an engagement means for coupling and holding components together. In a further aspect, the engagement means can be a screwing mechanism, a click-lock mechanism, or friction mechanism, or the like.

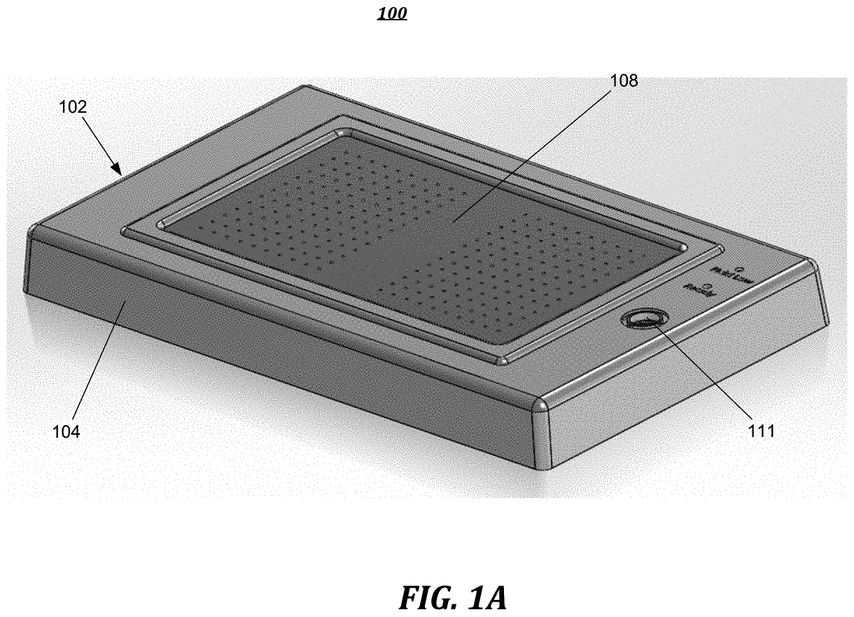

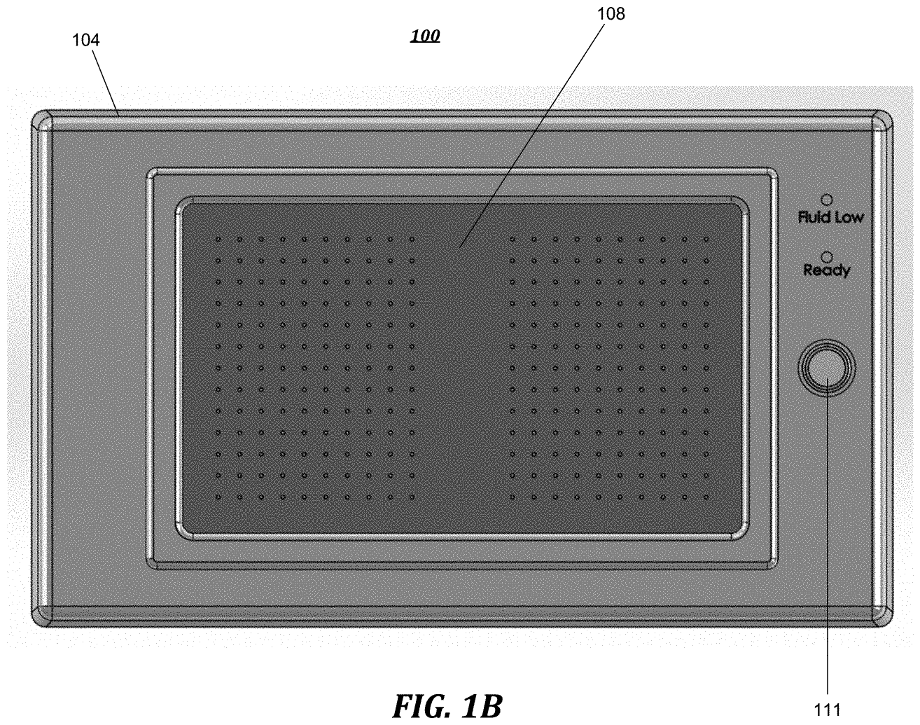

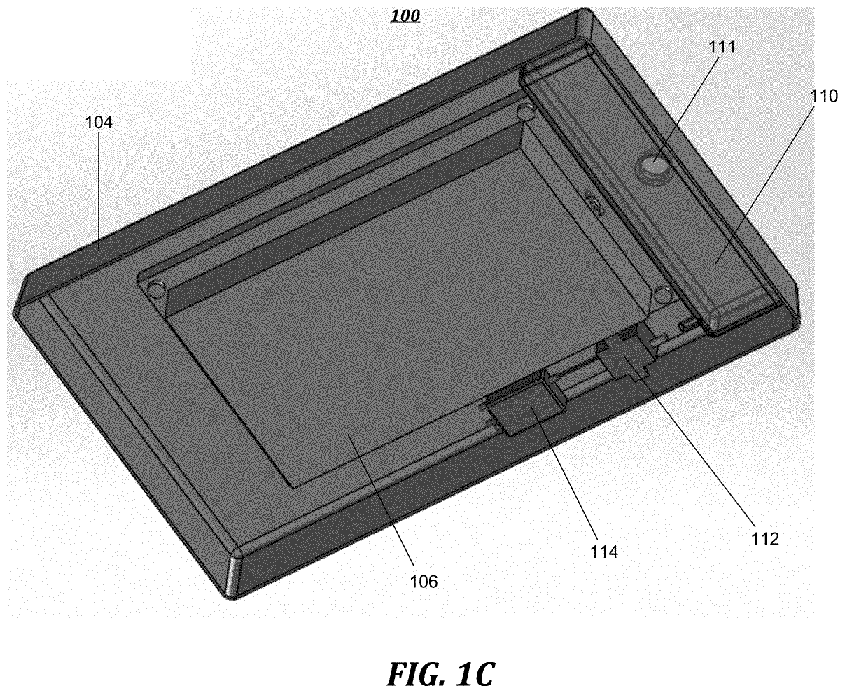

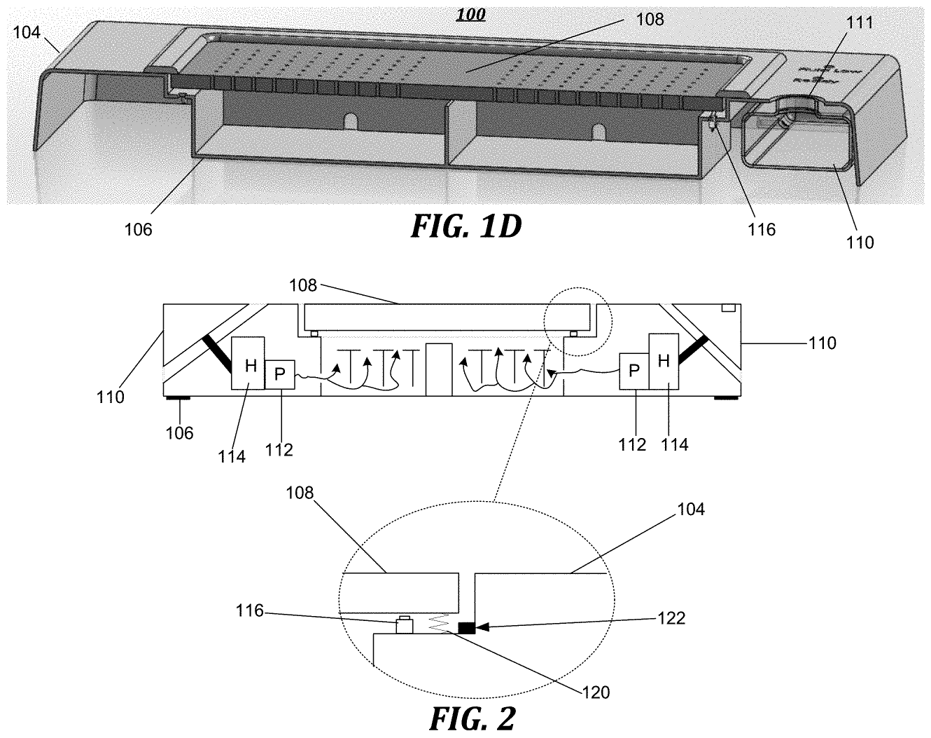

According to various aspects of the invention, the disclosed devices, systems, and methods can comprise multiple configurations. For example, aspects of various exemplary embodiments of the inventive sanitizing devices apparatuses, systems, and methods are shown in FIGS. 1A-6.

In one aspect, FIGS. 1A-1D depicts various view of sanitizing device 100 in accordance with an exemplary embodiment of the present disclosure. Sanitizing device 100 comprises housing 102, including upper housing frame 104 and lower housing frame 106, sanitizing platform 108, steam source system including steam source tank 110 with fill hole 111, pump 112, and heater 114; and activating switch 116 in operable communication with the steam source system, the switch being configured to selectively dispense a predetermined volume of steam (and/or sanitizing agent) through sanitizing platform apertures. Sanitizing platform 108 generally comprising a planar shaped surface having perforated equally spaced apertures. The apertures are sized and dimensioned effective to allow releasing steam and/or sanitizing agent to be dispensed onto an object or article on the platform. The platform surface may include an elevated surface forming a raised barrier around the surface having perforated equally spaced apertures. FIG. 1C shows the underside of the sanitizing device, depicting lower housing frame 106, tank 110, pump 112, and heater 114. Tank 110 may be configured to hold at least one of water, sanitizing agent, disinfectant, and/or other chemicals or liquids. Tank 110 may further comprise smaller containers or sections wherein the sections are each configured to hold a different liquid such that they do not mix within the reservoir container. In another aspect, the tank may be configured such that the liquids and/or chemicals mix within the container prior to being heated and/or dispensed through the platform.

As shown in FIG. 1D, platform 108 may be positioned in the center portion of sanitizing device housing between the upper housing frame 104 and lower housing frame 106. Platform 108 is held above lower frame 106 and switch 116 using spring element (120, FIG. 2). To this end, when a user step on the platform, user's weight will push down the platform down to engage with lower housing frame 106 and switch 116 to activate the sanitization process. Sanitization device 100 may further comprise a fluid indicator component and one more status indicator components, such as a LED. The activation switch may be at least one of a button, a switch, a pressure mechanism, a knob, a sensor, and the like.

FIG. 2 illustrates an alternative configuration having multiple tanks 110. In some embodiment, each tank may comprise different liquids and work independent of one another, and/or in tandem, depending on a user selection. Each tank may further contain section that may be configured to hold a different liquid such that they may or may not mix within the tank. In another aspect, the tank may be configured such that the liquids and/or chemicals mix within the tank prior to being heated and/or released through the planform. For example, one tank may comprise a first liquid, such as water, and configured to dispense steam, and the other tank may comprise a second liquid, such as a sanitizing agent, and configured to spray or atomize the second liquid through the platform. The steam and second liquid may be dispensed simultaneously and/or sequentially. In some embodiments, hard stop 122 may be included to stop downward movement of the platform.

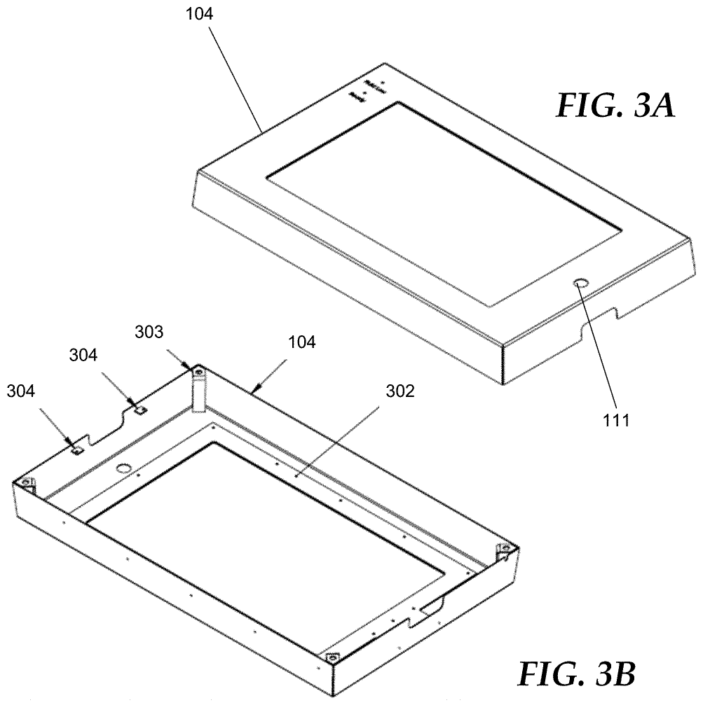

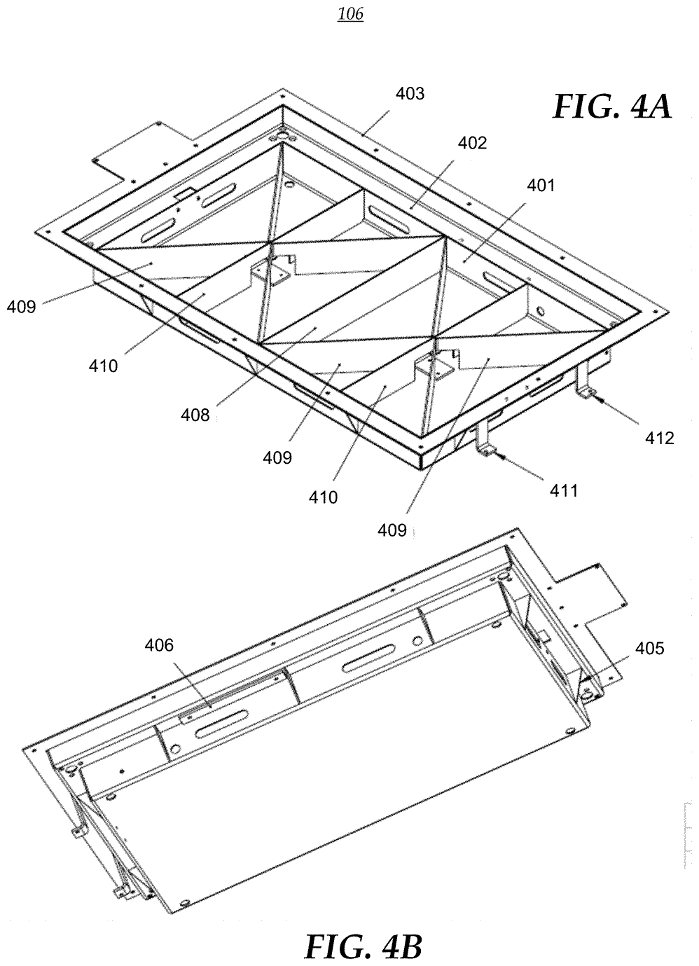

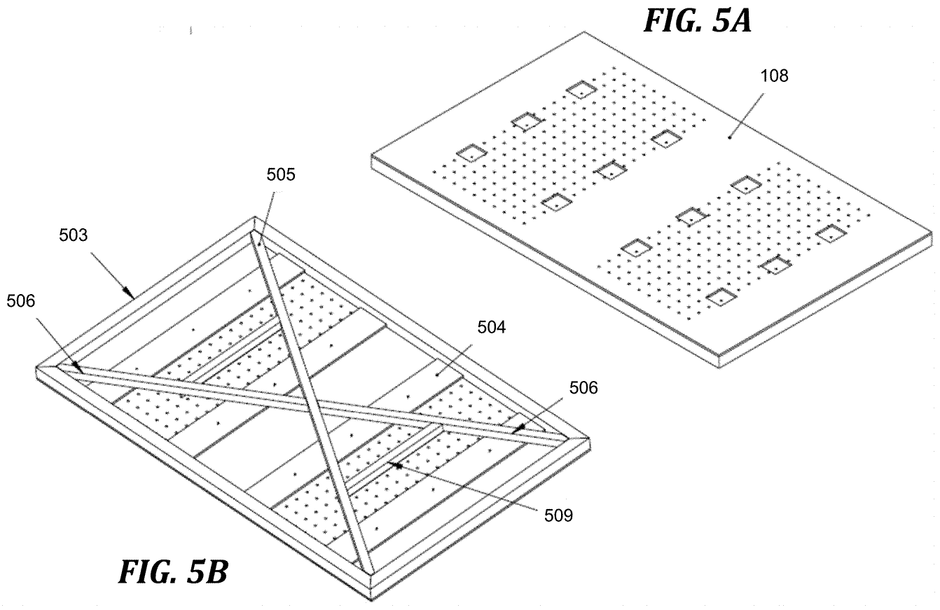

FIGS. 3A-3B depicts various views of upper housing frame 104 of sanitizing device 100. As shown, upper housing frame 104 includes mounting plate 302, mounting feet 303, tank mount 303, and fill hole 111. FIGS. 4A-4B depicts various views of lower housing frame 106 of sanitizing device 100. As shown, lower housing frame 104 includes lower pan 401, upper pan 402, bezel 403, nozzle mount 404, gusset 405, heater bracket 406, nozzle plate 407, platform center support 408, platform angle support 409, platform straight support 410, left tank support 411, and right tank support 412. Lower housing frame may comprise a plurality of apertures in the side walls of lower pan and platform supports to allow for supply lines, such as tubing, to travel from tanks and cover the entire lower pan area corresponding to area where the apertures are located in the platform above. In embodiments with multiple tanks, separate supply lines from each tank would be present and allow for dispending of steam from one tank through a first supply line and sanitizing agent from another tank through a second supply line. FIGS. 5A-5B depicts various views of sanitizing platform 108 of sanitizing device 100. Sanitizing platform 108 includes a plurality of apertures on the top surface, platform outer wall 503, platform cross walls 505, 506, platform cross support wall 509, and platform plate 504 on the bottom surface.

### Methods for Using the Devices and Systems

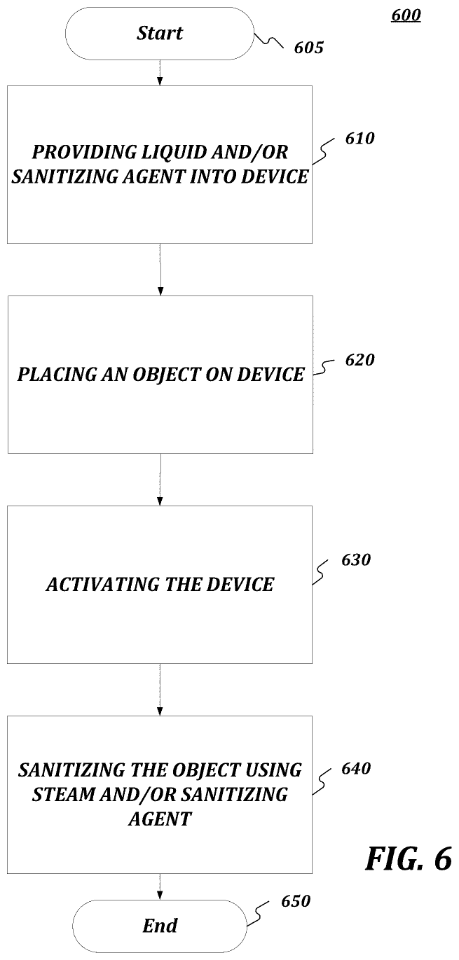

The present disclosure, according to further aspects, also provides methods of using the disclosed devices and systems. In one aspect, disclosed herein is a method for sanitizing an article of footwear. FIG. 6 shows a flow diagram of a sanitizing device, system and method of sanitizing objects in accordance with an exemplary embodiment of the present disclosure. Method 600 may begin at starting block 605 and proceed to stage 610 where the user may provide a liquid and/or sanitizing agent into sanitizing device. From stage 610, where the user of the sanitizing device provides a liquid and/or sanitizing agent, method 600 may advance to stage 620 where the user may step on or place an object, such as footwear, onto the sanitizing platform of the sanitizing device. Once the user steps on or places an object onto the sanitizing device in stage 620, method 600 may continue to stage 630 where the sanitization process is initiated by activating the switch.

After activation of the sanitizing device in stage 630, method 600 may proceed to stage 640 where the sanitizing device may sanitize the object by dispensing steam, sanitizing agent, or a combination of sanitizing agent and steam for a duration and volume effective to sanitize or disinfect the object. Once the sanitizing device sanitizes the object in stage 640, method 600 may then end at stage 650.

In various embodiments, the sanitizing device, system and methods of sanitizing may utilize a sanitizing agent configured to kill at least 99% of microbes, mold, and bacteria, for example, at least 99.9 or 99.99%. In further aspects, the present disclosure may further be embodied as a method, comprising: inserting a sanitizing agent comprising a chemical disinfectant into a sanitizing device; placing an object onto the sanitizing device; activating the sanitizing device; and sanitizing the object using a combination of the chemical disinfectant and steam. The aforementioned method may further be configured wherein the object further comprises an article of footwear. The aforementioned method may further be configured wherein the article of footwear further comprises at least one of: a boot, a shoe, a sandal, a high heel shoe, a slipper, a cleated shoe, a work boot, a dress shoe, and a sneaker.

The present disclosure includes at least the following aspects: Aspect 1: A device for sanitizing an article, the device comprising: a steam source for producing steam; a sanitizing platform comprising a plurality of apertures configured to allow a predetermined amount steam or sanitizing agent, or a combination of the foregoing therethrough effective to sanitize the article; a switch in operable communication with the steam source, the switch configured to selectively dispense a predetermined volume of steam through the apertures of the sanitizing platform; and a housing for containing the steam source and sanitizing platform.

Aspect 2: The device of any preceding aspect, further comprising at least one container or compartment comprising a liquid and/or sanitizing agent, the container being in operable communication with the steam. Aspect 3: The device of any preceding aspect, further comprising a plurality of containers or compartment, each comprising a liquid or sanitizing agent. Aspect 4: The device of any preceding aspect, further comprising a steam supply container containing a first liquid and a sanitizing agent container containing a sanitizing agent. Aspect 5: The device of any preceding aspect, wherein a container is in operable communication with the steam. Aspect 6: The device of any preceding aspect, wherein steam source container and/or sanitizing container is in operable communication with the steam. Aspect 7: The device of any preceding aspect, wherein the switch is configured to control a flow rate of steam from a steam supply line in fluid communication with the steam source. Aspect 8: The device of any preceding aspect, wherein the switch is configured to control a flow rate of sanitizing agent from a sanitizing agent supply line in fluid communication with a sanitizing agent container. Aspect 9: The device of any preceding aspect, wherein the switch is configured to independently control a flow rate of steam from a steam supply line in fluid communication with the steam source, and a flow rate of sanitizing agent from a sanitizing agent supply line in fluid communication with a sanitizing agent container.

Aspect 10: The device of any preceding aspect, wherein the steam is effective to cause at least a portion of a sanitizing agent within the container to be drawn or mixed into the steam stream to form a mixed stream.

Aspect 11: The device of any preceding aspect, wherein the switch is configured to control flow by moving a valve position from a closed position to an open position.

Aspect 12: The device of any preceding aspect, further comprising a housing or casing for housing one or more device components. Aspect 13: The device of any preceding aspect, wherein the switch comprises an actuating mechanism. Aspect 14: The device of any preceding aspect, wherein the switch comprises a step-triggered switch in mechanical communication with the sanitizing platform.

Aspect 15: The device of any preceding aspect, wherein the sanitizing platform further comprises a detachable cleaning pad. Aspect 16: The device of any preceding aspect, wherein the device is self-regulating.

Aspect 17: The device of any preceding aspect, wherein the switch in operative communication with a valve for selectively moving the valve from at least one of: a closed position to an open position and an open position to a closed position to control steam flow from the steam source.

Aspect 18: The device of any preceding aspect, further comprising a housing or casing for containing the steam source and/or sanitizing agent container.

Aspect 19: The device of any preceding aspect, further comprising at least one supply line configured to contain and/or carry a stream of steam.

Aspect 20: The device of any preceding aspect, further comprising at least one supply line configured to contain and/or carry a stream of sanitizing agent.

Aspect 21: The device of any preceding aspect, further comprising at least one supply line configured to contain and/or carry a mixed stream of sanitizing agent and steam.

Aspect 22: The device of any preceding aspect, further comprising at least one sanitizing agent supply line configured to carry sanitizing agent from within the sanitizing agent container into the steam stream. Aspect 23: The device of any preceding aspect, wherein the supply line is in fluid communication with the steam source. Aspect 24: The device of any preceding aspect, wherein the supply line is in fluid communication with the steam source container or a sanitizing agent container, or a combination thereof. Aspect 25: The device of any preceding aspect, wherein a sanitizing agent supply line is in fluid communication with the sanitizing agent and/or steam stream. Aspect 26: The device of any preceding aspect, wherein the steam source comprises at least one of a vessel, bottle, cylinder, tank, and cartridge, and the like. Aspect 27: The device of any preceding aspect, wherein the steam source is contained with the device housing. Aspect 28: The device of any preceding aspect, wherein the steam source is detachably connected to the device. Aspect 29: The device of any preceding aspect, wherein the steam source container is detachably connected to the device.

Aspect 30: The device of any preceding aspect, wherein the sanitizing agent container is detachably connected to the device.

Aspect 31: The device of any preceding aspect, wherein the steam source comprises a chamber comprising at least one of a liquid or sanitizing agent; wherein the chamber is configured to contain steam released from a tank.

Aspect 32: The device of any preceding aspect, wherein the steam source comprises a chamber formed in a lower housing frame; wherein the first chamber is configured to contain steam released from a steam source container.

Aspect 33: The device of any preceding aspect, wherein the steam source is an external source remotely located from the device.

Aspect 34: The device of any preceding aspect, wherein the sanitizing agent container is detachably connected to the device.

Aspect 35: The device of any preceding aspect, wherein the sanitizing agent container is a cartridge or the like.

Aspect 36: The device of any preceding aspect, further comprising a flow control component in fluid communication with the steam stream, and configured to control a flow rate of a mixed steam/sanitizing agent stream.

Aspect 37: The device of any preceding aspect, wherein the flow control component is configured to control the steam flow rate.

Aspect 38: The device of any preceding aspect, wherein the sanitizing agent comprises at least one of: water, steam, a disinfecting composition, antimicrobial composition, antibacterial composition, bactericidal composition, bacteriostatic composition or the like.

Aspect 39: The device of any preceding aspect, wherein the device is configured to dispense steam and sanitizing agent sequentially through the sanitizing platform.

Aspect 40: The device of any preceding aspect, wherein the device is configured to dispense steam and sanitizing agent simultaneously through the sanitizing platform.

Aspect 41: The device of any preceding aspect, wherein the device is configured to dispense steam and sanitizing agent simultaneously as a mixed stream through the sanitizing platform.

Aspect 42: The device of any preceding aspect, wherein the device is configured to dispense steam and sanitizing agent simultaneously as independent streams through the sanitizing platform.

Aspect 43: A method comprising:

inserting a disinfecting composition or chemical disinfectant into a sanitizing device of any preceding aspect; placing an object onto the sanitizing device; activating the sanitizing device; and

sanitizing the object using a combination of the chemical disinfectant and steam.

Aspect 44: The method of any preceding aspect, wherein the article comprises an article of footwear. Aspect 45: The method of any preceding aspect, wherein the article of footwear further comprises at least one of: a boot, a shoe, a sandal, a high heel shoe, a slipper, a cleated shoe, a work boot, a dress shoe, and a sneaker.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application.

1. A device for sanitizing an article, the device comprising:
a. a steam source for producing steam;
b. a sanitizing platform comprising a plurality of apertures configured to allow a predetermined amount steam or sanitizing agent, or a combination of the foregoing therethrough effective to sanitize the article;
c. a switch in operable communication with the steam source, the switch configured to selectively dispense a predetermined volume of steam through the apertures of the sanitizing platform; and
d. a housing for containing the steam source and sanitizing platform.

2. The device of claim 1, further comprising at least one container or compartment comprising a liquid or a sanitizing agent, the container being in operable communication with the steam.

3. The device of claim 2, further comprising a plurality of containers or compartment, each comprising a liquid or sanitizing agent.

4. The device of claim 3, further comprising wherein the plurality of containers comprise a steam supply container containing a first liquid and a sanitizing agent container containing a sanitizing agent.

5. (canceled)

6. (canceled)

7. The device of claim 4, wherein the switch is configured to control a flow rate of steam from a steam supply line in fluid communication with the steam source.

8. The device of claim 7, wherein the switch is configured to control a flow rate of sanitizing agent from a sanitizing agent supply line in fluid communication with a sanitizing agent container.

9. The device of claim 8, wherein the switch is configured to independently control a flow rate of steam from a steam supply line in fluid communication with the steam source, and a flow rate of sanitizing agent from a sanitizing agent supply line in fluid communication with a sanitizing agent container.

10. The device of claim 9, wherein the steam is effective to cause at least a portion of a sanitizing agent within the container to be drawn or mixed into the steam stream to form a mixed stream.

11. (canceled)

12. (canceled)

13. (canceled)

14. The device of claim 9, wherein the switch comprises a step-triggered switch in mechanical communication with the sanitizing platform.

15. The device of claim 14, wherein the sanitizing platform further comprises a detachable cleaning pad.

16. (canceled)

17. (canceled)

18. (canceled)

19. (canceled)

20. (canceled)

21. The device of claim 10, further comprising at least one supply line configured to contain and/or carry a mixed stream of sanitizing agent and steam.

22. (canceled)

23. (canceled)

24. (canceled)

25. (canceled)

26. (canceled)

27. (canceled)

28. (canceled)

29. (canceled)

30. The device of claim 14, wherein the sanitizing agent container is detachably connected to the device.

31. (canceled)

32. The device of claim 14, wherein the steam source comprises a chamber formed in a lower housing frame; wherein the first chamber is configured to contain steam released from a steam source container.

33. (canceled)

34. (canceled)

35. (canceled)

36. The device of claim 21, further comprising a flow control component in fluid communication with the steam stream, and configured to control a flow rate of a mixed steam/sanitizing agent stream.

37. (canceled)

38. (canceled)

39. The device of claim 32, wherein the device is configured to dispense steam and sanitizing agent sequentially through the sanitizing platform.

40. The device of claim 32, wherein the device is configured to dispense steam and sanitizing agent simultaneously through the sanitizing platform.

41. The device of claim 36, wherein the device is configured to dispense steam and sanitizing agent simultaneously as a mixed stream through the sanitizing platform.

42. The device of claim 40, wherein the device is configured to dispense steam and sanitizing agent simultaneously as independent streams through the sanitizing platform.

43. A method comprising:
inserting a disinfecting composition or chemical disinfectant into a sanitizing device of any preceding claim 42;
placing an object onto the sanitizing device;
activating the sanitizing device; and
sanitizing the object using a combination of the chemical disinfectant and steam.

44. The method of claim 43, wherein the article comprises an article of footwear.

45. (canceled)

46. The method of claim 44, wherein activating comprises stepping on the step-triggered switch in mechanical communication with the sanitizing platform; and sanitizing comprises dispensing steam and sanitizing agent simultaneously as independent streams through the sanitizing platform effective to sanitize the object.
